(19) United States
(12) Patent Application Publication
(10) Pub. No.: US 2026/0033981 A1
(43) Pub. Date: Feb. 5, 2026

(54) THERMAL THERAPY SYSTEM

(71) Applicant: Avent, Inc., Alpharetta, GA (US)

(72) Inventors: Jeffrey Broadrick, Alpharetta, GA (US); Bryan D. Huff, Alpharetta, GA (US); Christian Erdman, Alpharetta, GA (US); Shawn Michael Brogdon, Alpharetta, GA (US)

(21) Appl. No.: 19/290,660

(22) Filed: Aug. 5, 2025

Related U.S. Application Data
(60) Provisional application No. 63/679,204, filed on Aug. 5, 2024.

(51) Int. Cl. A61F 7/00; A61F 7/02

(52) U.S. Cl. CPC A61F 7/0053; A61F 7/02; A61F 2007/0056; A61F 2007/0093

(57) ABSTRACT

A cold compression system may include a housing including a compressor outlet, a coolant outlet, a cavity, and an internal coolant port in fluid communication with the coolant outlet. A fluid compressor is arranged in fluid communication with the compressor outlet. A positive displacement fluid pump is arranged in fluid communication between the internal coolant port and the coolant outlet. A controller is structured to selectively activate the fluid compressor or the positive displacement fluid pump. A removable fluid reservoir is sized to fit within the cavity and includes a reservoir defining a water fill line, and a reservoir port positioned above the water fill line and structured to couple with the internal coolant port of the housing. A cold compression pad includes a compression chamber coupled to the compressor outlet of the housing, and a heat exchanger coupled to the coolant outlet of the housing.

### CROSS-REFERENCE TO RELATED APPLICATION

This application claims the benefit of U.S. Provisional Application No. 63/679,204, filed Aug. 5, 2024, the contents of which are incorporated herein by reference in its entirety.

### BACKGROUND

This disclosure is related to thermal therapy devices. More particularly, this disclosure is related to thermal therapy devices that can be filled with ice and water.

### SUMMARY

In some aspects, the techniques described herein relate to a cold compression system including: a housing including a compressor outlet, a coolant outlet, a cavity, and an internal coolant port in fluid communication with the coolant outlet; a fluid compressor supported by the housing and arranged in fluid communication with the compressor outlet; a positive displacement fluid pump supported by the housing and arranged in fluid communication between the internal coolant port and the coolant outlet; one or more processing circuits supported by the housing and including one or more memory devices coupled to one or more processors, the one or more memory devices configured to store instructions thereon that, when executed by the one or more processors, cause the one or more processors to selectively activate the fluid compressor or the positive displacement fluid pump; a removable fluid reservoir sized to fit within the cavity, the removable fluid reservoir including: a reservoir defining a water fill line, and a reservoir port positioned above the water fill line and structured to selectively fluidly couple with the internal coolant port of the housing; and a cold compression pad including a compression chamber selectively fluidly coupled to the compressor outlet of the housing, and a heat exchanger selectively fluidly coupled to the coolant outlet of the housing.

In some aspects, the techniques described herein relate to a cold compression system, further including a user interface in communication with the one or more processing circuits, wherein the one or more memory devices are further configured to store instructions thereon that, when executed by the one or more processors, cause the one or more processors to: receive pressure information from the user interface; control operation of the fluid compressor based on the pressure information; receive temperature information from the user interface; and control operation of the positive displacement fluid pump based on the temperature information.

In some aspects, the techniques described herein relate to a cold compression system, wherein the housing includes thermal insulation adjacent the cavity.

In some aspects, the techniques described herein relate to a cold compression system, wherein the housing includes a housing carry handle.

In some aspects, the techniques described herein relate to a cold compression system, wherein the removable fluid reservoir includes a reservoir carry handle.

In some aspects, the techniques described herein relate to a cold compression system, wherein the housing includes a reservoir handle recess sized to receive the reservoir carry handle.

In some aspects, the techniques described herein relate to a cold compression system, wherein the housing includes a housing carry handle movable between a housing handle stowed position adjacent a first side of the housing and a housing handle deployed position, and wherein the removable fluid reservoir includes a reservoir carry handle movable between a reservoir handle stowed position adjacent a second side of the housing opposite the first side of the housing and a reservoir handle deployed position.

In some aspects, the techniques described herein relate to a cold compression system, wherein the housing includes a housing projection and the internal coolant port is positioned on the housing projection, and wherein the removable fluid reservoir includes a reservoir recess sized to receive the housing projection and the reservoir port is positioned on the reservoir recess.

In some aspects, the techniques described herein relate to a cold compression system, wherein the housing includes a housing projection, and wherein the removable fluid reservoir includes a reservoir recess sized to receive the housing projection and inhibit movement of the removable fluid reservoir within the housing.

In some aspects, the techniques described herein relate to a cold compression system, wherein at least one of the internal coolant port or the reservoir port includes a valve that allows fluid flow when the internal coolant port and the reservoir port are coupled together and inhibit flow when the internal coolant port and the reservoir port are decoupled.

In some aspects, the techniques described herein relate to a cold compression system, wherein the internal coolant port engages the reservoir port vertically along a valve axis.

In some aspects, the techniques described herein relate to a cold compression system, wherein the removable fluid reservoir includes a pickup tube fluidly coupled to the reservoir port and extending below the water fill line.

In some aspects, the techniques described herein relate to a cold compression system, wherein the pickup tube is formed within the reservoir.

In some aspects, the techniques described herein relate to a cold compression system, wherein the removable fluid reservoir includes a lid selectively covering the reservoir.

In some aspects, the techniques described herein relate to a cold compression system, wherein the removable fluid reservoir is removable from the housing with the cold compression pad connected to the housing.

In some aspects, the techniques described herein relate to a system including: a housing including a compressor outlet, a coolant outlet, a cavity, and an internal coolant port in fluid communication with the coolant outlet; a fluid compressor supported by the housing and arranged in fluid communication with the compressor outlet; a positive displacement fluid pump supported by the housing and arranged in fluid communication between the internal coolant port and the coolant outlet; one or more processing circuits supported by the housing and including one or more memory devices coupled to one or more processors, the one or more memory devices configured to store instructions thereon that, when executed by the one or more processors, cause the one or more processors to selectively activate the fluid compressor or the positive displacement fluid pump; and a removable fluid reservoir sized to fit within the cavity, the removable fluid reservoir including: a reservoir defining a water fill line, and a reservoir port positioned above the water fill line and structured to selectively fluidly couple with the internal coolant port of the housing.

In some aspects, the techniques described herein relate to a system, wherein at least one of the internal coolant port or the reservoir port includes a valve that allows fluid flow when the internal coolant port and the reservoir port are coupled together and inhibit flow when the internal coolant port and the reservoir port are decoupled.

In some aspects, the techniques described herein relate to a system, wherein the removable fluid reservoir includes a pickup tube fluidly coupled to the reservoir port and extending below the water fill line.

In some aspects, the techniques described herein relate to a system, wherein the housing includes a housing projection and the internal coolant port is positioned on the housing projection, and wherein the removable fluid reservoir includes a reservoir recess sized to receive the housing projection and the reservoir port is positioned on the reservoir recess.

In some aspects, the techniques described herein relate to a bucket configured for use with a cold compression device, the bucket including: at least one side wall including an insulation material; a reservoir defined by the at least one side wall the reservoir further defining a water fill line; and a reservoir port positioned above the water fill line and structured to selectively fluidly couple with an internal coolant port of a housing of the cold compression device.

In some aspects, the techniques described herein relate to a bucket, wherein the bucket further includes a pickup tube fluidly coupled to the reservoir port and extending below the water fill line.

In some aspects, the techniques described herein relate to a bucket, wherein the pickup tube is formed within the at least one side wall.

In some aspects, the techniques described herein relate to a bucket, wherein the bucket includes a reservoir carry handle movable between a reservoir handle stowed position adjacent a first side of the housing opposite a second side of the housing and a reservoir handle deployed position.

This summary is illustrative only and is not intended to be in any way limiting. Other aspects, features, and advantages of the devices or processes described herein will become apparent in the detailed description set forth herein, taken in conjunction with the accompanying figures, wherein like reference numerals refer to like elements.

### BRIEF DESCRIPTION OF DRAWINGS

The device is explained in even greater detail in the following drawings. The drawings are merely exemplary and certain features may be used singularly or in combination with other features. The drawings are not necessarily drawn to scale.

FIG. 1 is a schematic diagram of a thermal therapy system, according to some implementations.

FIG. 2 is a schematic diagram of a controller of the thermal therapy system of FIG. 1, according to some implementations.

FIG. 3 is a perspective view of the thermal therapy system of FIG. 1 including a housing, a removable fluid reservoir, and a cold compression pad, according to some implementations.

FIG. 4 is a perspective view of the thermal therapy system of FIG. 3 with a housing handle and a reservoir handle partially raised, according to some implementations.

FIG. 5 is a front view of the thermal therapy system of FIG. 3.

FIG. 6 is a top view of the thermal therapy system of FIG. 3.

FIG. 7 is a side view of the thermal therapy system of FIG. 3.

FIG. 8 is a perspective view of the thermal therapy system of FIG. 3 with the removable fluid reservoir removed from the cavity of the housing, according to some implementations.

FIG. 9 is a sectional view of the thermal therapy system of FIG. 3, according to some implementations.

FIG. 10 is a perspective view of a fluid reservoir, according to some implementations.

FIG. 11 is a sectional view of the fluid reservoir of FIG. 11, according to some implementations.

### DETAILED DESCRIPTION

Following below are more detailed descriptions of concepts related to, and implementations of, methods, apparatuses, and systems for a thermal therapy system. The figures illustrate exemplary implementations in detail and the present disclosure is not limited to the details or methodology set forth in the description or illustrated in the figures. The terminology used herein is for the purpose of description only and should not be regarded as limiting.

Referring to the figures generally, the various implementations disclosed herein relate to systems, apparatuses, and methods for a thermal therapy system (e.g., a cold compression system) that includes a reservoir or bucket and pumps fluid (e.g., ice water) through a compression pad or wrap to provide treatment to a user. The reservoir is removable from the system so that it can be easily filled with ice and water without necessitating moving the entire thermal therapy system.

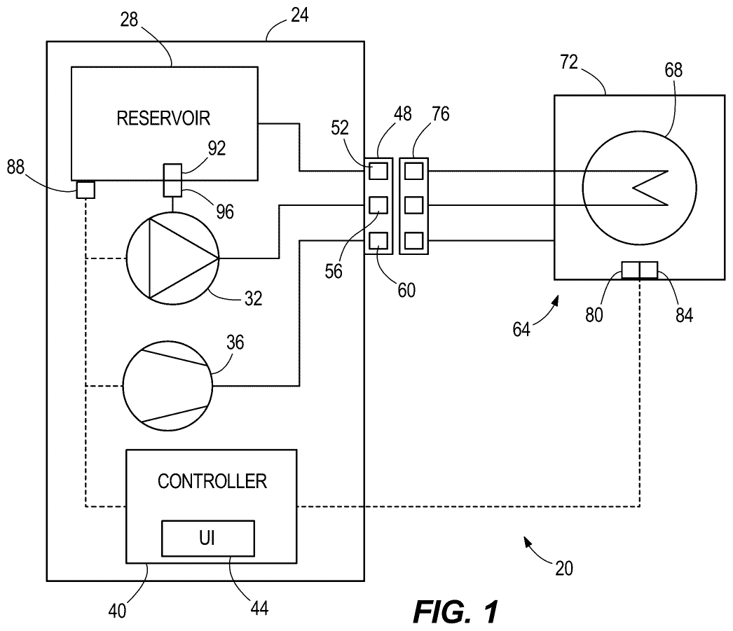

As shown in FIG. 1, a thermal therapy system in the form of a cold compression system 20 includes a housing 24 supporting a removable fluid reservoir 28, a positive displacement fluid pump 32, a fluid compressor 36, and a controller 40 including a user interface 44. In some implementations, the positive displacement fluid pump 32 is a diaphragm pump and the fluid compressor 36 is an air compressor. Generally, the removable fluid reservoir 28 is removable from the removable fluid reservoir 28 to allow a user to fill the removable fluid reservoir 28 with water and/or ice at a location remote from the housing 24. The housing 24 also supports a unit connector 48 including a reservoir return port 52, a coolant outlet in the form of a pump out port 56, and a compressor outlet in the form of a compressor out port 60.

A therapy pad in the form of a cold compressor pad 64 includes a heat exchanger 68 and a compression chamber 72 that receives and holds pressure from the fluid compressor 36. The cold compressor pad 64 includes a pad connector 76 corresponding to the unit connector 48 that is connected to the heat exchanger 68 and the compression chamber 72 via flexible lines.

In some implementations, the cold compressor pad 64 includes a pressure sensor 80 and a temperature sensor 84 arranged in communication (e.g., wired, wireless, etc.) with the controller 40. In some implementations, the pressure sensor 80 and the temperature sensor 84 are positioned within the housing 24. For example, the pressure sensor 80 can be positioned to monitor a pressure leaving the fluid compressor 36 or at the port 60 of the unit connector 48. For example, the temperature sensor 84 can be positioned to monitor a temperature of fluid leaving the positive displacement fluid pump 32, entering the pump out port 56, or returning through the reservoir return port 52. The temperature sensor 84 could also be positioned to monitor a temperature of the fluid inside the removable fluid reservoir 28. In some implementations, more than one pressure sensor and/or more than one temperature sensor is included. In some implementations, a reservoir sensor 88 determines the presence or absence of the removable fluid reservoir 28 within the housing 24 and inhibits operation of the positive displacement fluid pump 32 and/or the fluid compressor 36 when the removable fluid reservoir 28 is not installed within the housing 24.

In some implementations, the housing 24 and the removable fluid reservoir 28 include an internal coolant port including a reservoir port 92 and a housing port 96. The reservoir port 92 and the housing port 96 are selectively couplable to one another to form a watertight seal and allow flow from the removable fluid reservoir 28 to the positive displacement fluid pump 32. In some implementations, the reservoir port 92 includes a valve. In some implementations the housing port 96 includes a valve. In some implementations, the internal coolant port only allows fluid flow when the reservoir port 92 is fluidly coupled to the housing port 96. In some implementations, the reservoir port 92 and the housing port 96 are arranged relative to one another so that engagement of the reservoir port 92 with the housing port 96 so that engagement occurs along a vertical valve axis.

In some implementations, a return from the reservoir return port 52 provides water to the removable fluid reservoir 28 above a water line. In some implementations, the return from the reservoir return port 52 is connected to the internal coolant port.

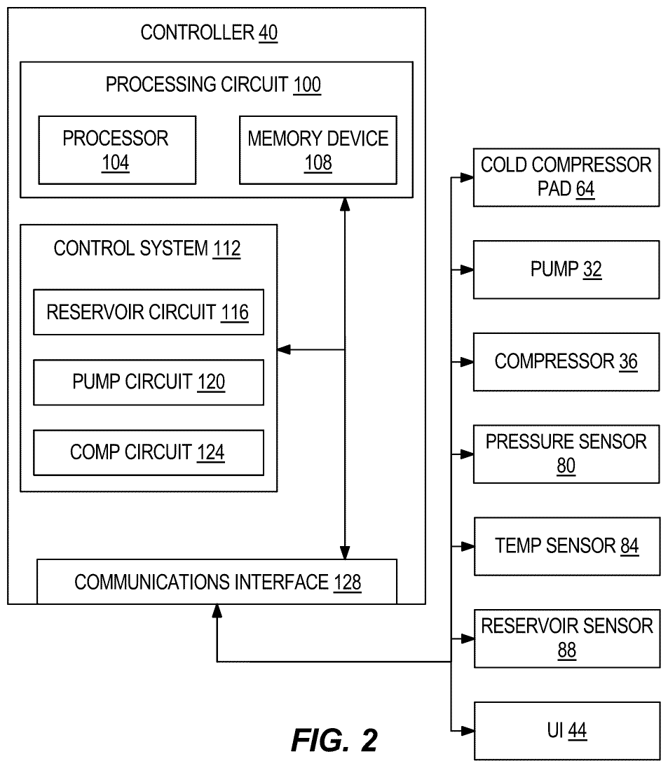

Referring now to FIG. 2, a schematic diagram of the controller 40 is shown according to an example implementation. As shown in FIG. 2, the controller 40 includes a processing circuit 100 having a processor 104 and a memory device 108, a control system 112 having a reservoir circuit 116, a pump circuit 120, and a compressor circuit 124, and a communications interface 128. Generally, the controller 40 is structured to control operation of the positive displacement fluid pump 32 and the fluid compressor 36 to provide desired therapies to the user via the cold compressor pad 64. The user can interact with the controller 40 via the user interface 44 to select desired temperatures, pressures, etc.

In one configuration, the circuits of the control system 112 are in the form of machine or computer-readable media that is executable by a processor, such as processor 104. As described herein, the machine-readable media facilitates performance of certain operations to enable reception and transmission of data. For example, the machine-readable media may provide an instruction (e.g., command, etc.) to acquire data. In this regard, the machine-readable media may include programmable logic that defines the frequency of acquisition of the data (or, transmission of the data). The computer readable media may include code written in any programming language. The computer readable program code may be executed on one processor, multiple co located processors, multiple remote processors, or any combination of local and remote processors. Remote processors may be connected to each other through any type of network (e.g., CAN bus, etc.).

In another configuration, the circuits of the control system 112 are implemented as hardware units, such as electronic control units. As such, the circuits of the control system 112 may be implemented as one or more circuitry components including, but not limited to, processing circuitry, network interfaces, peripheral devices, input devices, output devices, sensors, etc. In some implementations, the circuits of the control system 112 may take the form of one or more analog circuits, electronic circuits (e.g., integrated circuits (IC), discrete circuits, system on a chip (SOCs) circuits, microcontrollers, etc.), telecommunication circuits, hybrid circuits, and any other type of “circuit.” In this regard, the circuits of the control system 112 may include any type of component for accomplishing or facilitating achievement of the operations described herein. For example, a circuit as described herein may include one or more transistors, logic gates (e.g., NAND, AND, NOR, OR, XOR, NOT, XNOR, etc.), resistors, multiplexers, registers, capacitors, inductors, diodes, wiring, and so on). The circuits of the control system 112 may also include programmable hardware devices such as field programmable gate arrays, programmable array logic, programmable logic devices or the like. The circuits of the control system 112 may include one or more memory devices for storing instructions that are executable by the processor(s) of the circuits of the control system 112. The one or more memory devices and processor(s) may have the same definition as provided below with respect to the memory device 108 and processor 104. In some hardware unit configurations, the circuits of the control system 112 may be geographically dispersed throughout separate locations. Alternatively and as shown, the circuits of the control system 112 may be implemented in or within a single unit/housing, which is shown as the controller 40.

In the example shown, the controller 40 includes the processing circuit 100 having the processor 104 and the memory device 108. The processing circuit 100 may be structured or configured to execute or implement the instructions, commands, and/or control processes described herein with respect to the circuits of the control system 112. The depicted configuration represents the circuits of the control system 112 as machine or computer-readable media. However, as mentioned above, this illustration is not meant to be limiting as the present disclosure contemplates other implementations where the circuits of the control system 112, or at least one circuit of the circuits of the control system 112, is configured as a hardware unit. All such combinations and variations are intended to fall within the scope of the present disclosure.

The hardware and data processing components used to implement the various processes, operations, illustrative logics, logical blocks, modules and circuits described in connection with the implementations disclosed herein (e.g., the processor 104) may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, or state machine. A processor also may be implemented as a combination of computing devices, such as a combination of a DSP and a microprocessor, a plurality of microprocessors, one or more microprocessors in conjunction with a DSP core, or any other such configuration. In some implementations, the one or more processors may be shared by multiple circuits (e.g., the circuits of the control system 112 may comprise or otherwise share the same processor which, in some example implementations, may execute instructions stored, or otherwise accessed, via different areas of memory). Alternatively or additionally, the one or more processors may be structured to perform or otherwise execute certain operations independent of one or more co-processors. In other example implementations, two or more processors may be coupled via a bus to enable independent, parallel, pipelined, or multi-threaded instruction execution. All such variations are intended to fall within the scope of the present disclosure.

The memory device 108 (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory device 108 may be communicably connected to the processor 104 to provide computer code or instructions to the processor 104 for executing at least some of the processes described herein. Moreover, the memory device 108 may be or include tangible, non-transient volatile memory or non-volatile memory. Accordingly, the memory device 108 may include database components, object code components, script components, or any other type of information structure for supporting the various activities and information structures described herein.

The reservoir circuit 116 is structured to determine the presence or absence of the removable fluid reservoir 28 via the reservoir sensor 88. In some implementations, the reservoir circuit 116 receives information from the reservoir sensor 88 and inhibits operation of the positive displacement fluid pump 32 and/or the fluid compressor 36 if the removable fluid reservoir 28 is not installed in the housing 24. In some implementations the reservoir sensor 88 includes a microswitch and the information provided by the reservoir sensor 88 is a sense voltage. For example, if the reservoir circuit 116 detects OV, then the removable fluid reservoir 28 is determined to be removed from the housing 24 and operation of the positive displacement fluid pump 32 and the fluid compressor 36 is inhibited.

The pump circuit 120 is structured to operate the positive displacement fluid pump 32. In some implementations, the pump circuit 120 receives temperature information from the temperature sensor 84 and operates the positive displacement fluid pump 32 to maintain a desired temperature input into the user interface 44 by the user. In some implementations, the pump circuit 120 receives a PUMP-ON signal from the user interface 44 and operates the positive displacement fluid pump 32 continually until a PUMP-OFF signal is received from the user interface 44.

The compressor circuit 124 is structured to operate the fluid compressor 36. In some implementations, the compressor circuit 124 receives pressure information from the pressure sensor 80 and operates the fluid compressor 36 to maintain a desired pressure input into the user interface 44 by the user. In some implementations, the compressor circuit 124 receives a PRESSURE-ON signal form the user interface 44 and operates the fluid compressor 36 to maintain a predetermine pressure until a PRESSURE-OFF signal is received from the user interface 44.

While various circuits with particular functionality are shown in FIG. 2, it should be understood that the controller 40 may include any number of circuits for completing the functions described herein. For example, the activities and functionalities of the circuits of the control system 112 may be combined in multiple circuits or as a single circuit. Additional circuits with additional functionality may also be included. Further, the controller 40 may further control other activity beyond the scope of the present disclosure. In some implementations, the circuits described herein may include one or more processing circuits comprising one or more memory devices coupled to one or more processors, the one or more memory devices configured to store instructions thereon that, when executed by the one or more processors, cause the one or more processors to perform the operations performed herein and described with reference to circuits.

As mentioned above and in one configuration, the “circuits” may be implemented in machine-readable medium for execution by various types of processors, such as the processor 104 of FIG. 2. An identified circuit of executable code may, for instance, comprise one or more physical or logical blocks of computer instructions, which may, for instance, be organized as an object, procedure, or function. Nevertheless, the executables of an identified circuit need not be physically located together, but may comprise disparate instructions stored in different locations which, when joined logically together, comprise the circuit and achieve the stated purpose for the circuit. Indeed, a circuit of computer readable program code may be a single instruction, or many instructions, and may even be distributed over several different code segments, among different programs, and across several memory devices. Similarly, operational data may be identified and illustrated herein within circuits, and may be implemented in any suitable form and organized within any suitable type of data structure. The operational data may be collected as a single data set, or may be distributed over different locations including over different storage devices, and may exist, at least partially, merely as electronic signals on a system or network.

While the term “processor” is briefly defined above, the term “processor” and “processing circuit” are meant to be broadly interpreted. In this regard and as mentioned above, the “processor” may be implemented as one or more general-purpose processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some implementations, the one or more processors may be external to the apparatus, for example the one or more processors may be a remote processor (e.g., a cloud based processor). Alternatively or additionally, the one or more processors may be internal and/or local to the apparatus. In this regard, a given circuit or components thereof may be disposed locally (e.g., as part of a local server, a local computing system, etc.) or remotely (e.g., as part of a remote server such as a cloud based server). To that end, a “circuit” as described herein may include components that are distributed across one or more locations.

Implementations within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

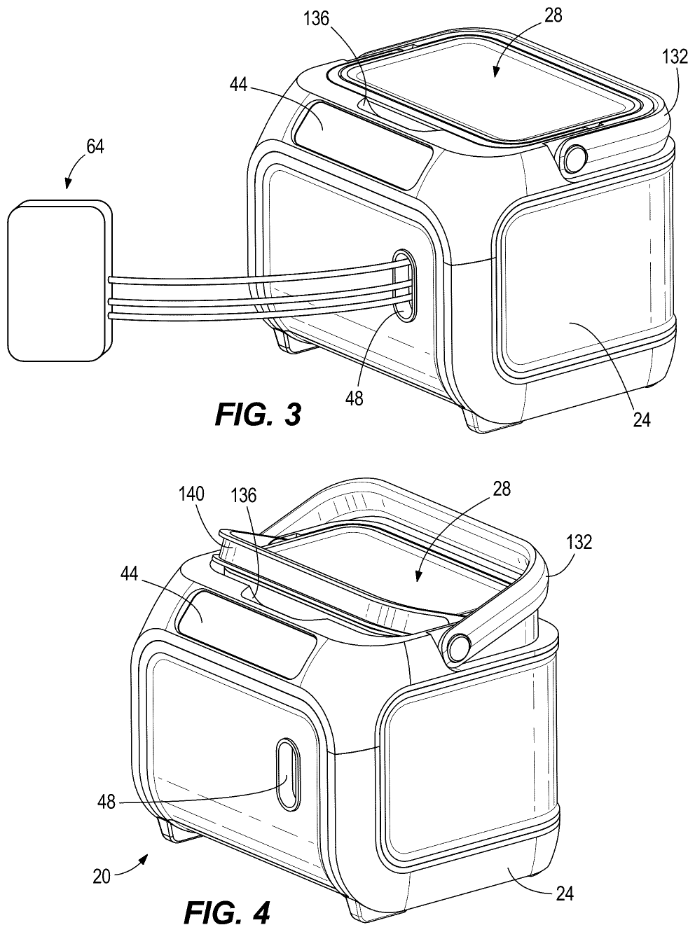

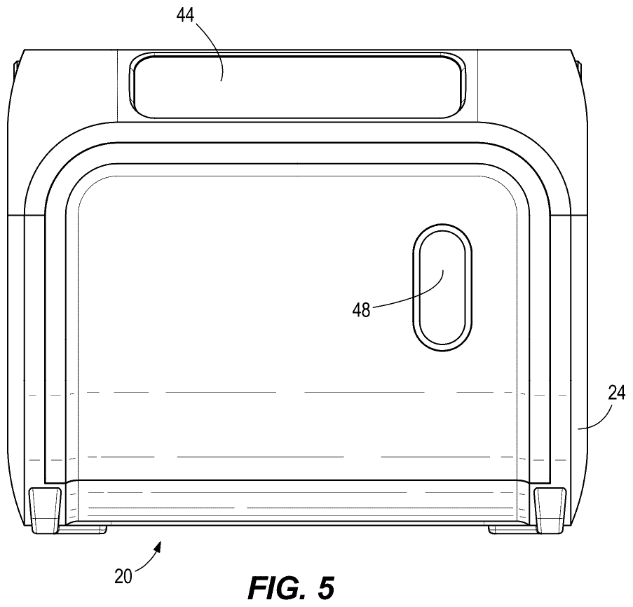

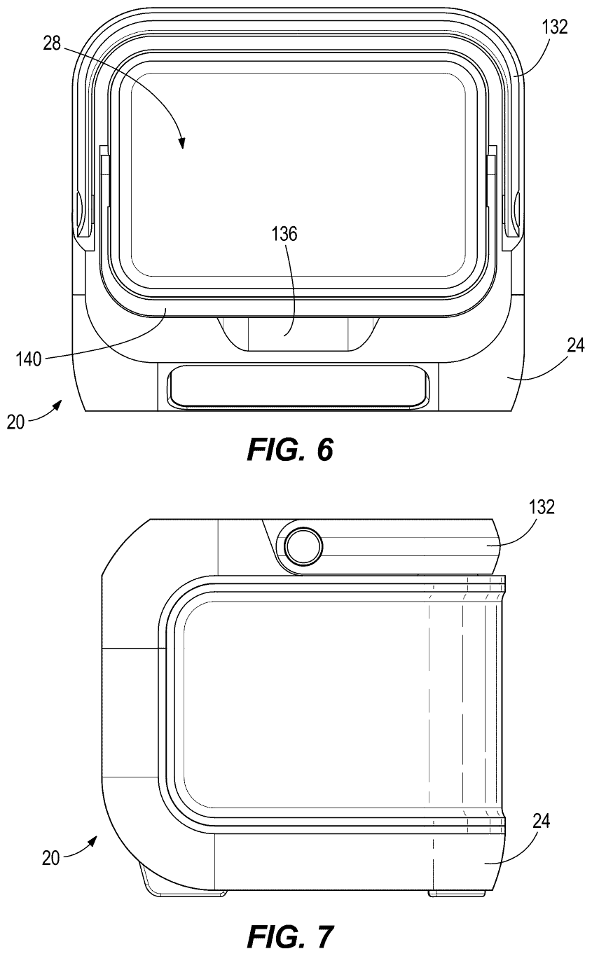

As shown in FIGS. 3-7, some implementations of the cold compression system 20 include a molded plastic housing 24 including assembled sides and feet. A housing carry handle 132 is pivotably connected to the housing 24 and structured to support the weight of the cold compression system 20 even when filled with fluid. In some implementations, the housing carry handle 132 is a u-shaped handle. The housing carry handle 132 is movable between a housing handle stowed position (see FIG. 3) adjacent a first side of the housing and a housing handle deployed position (see FIG. 4).

The housing 24 also includes a hand recess 136 sized to provide access to the removable fluid reservoir 28 to aid in removal of the removable fluid reservoir 28 from the housing 24. The user interface 44 is visible through the housing 24 or extends through the housing 24 so that the user can interact with the user interface 44.

As shown in FIG. 3, the unit connector 48 is arranged on a front side of the housing 24 to provide easy access to the user and make connection of the cold compressor pad 64 to the unit connector 48 via the pad connector 76 simple. In some implementations, the unit connector 48 is asymmetrical so that the pad connector 76 can only be installed in the desired orientation.

As shown in FIG. 4, the removable fluid reservoir 28 includes a reservoir carry handle 140 movable between a reservoir handle stowed position (see FIG. 3) adjacent a second side of the housing 24 opposite the first side of the housing 24 and a reservoir handle deployed position (see FIG. 4). The reservoir carry handle 140 is pivotably connected to the removable fluid reservoir 28 and is structured to support the weight of the removable fluid reservoir 28 when filled with fluid.

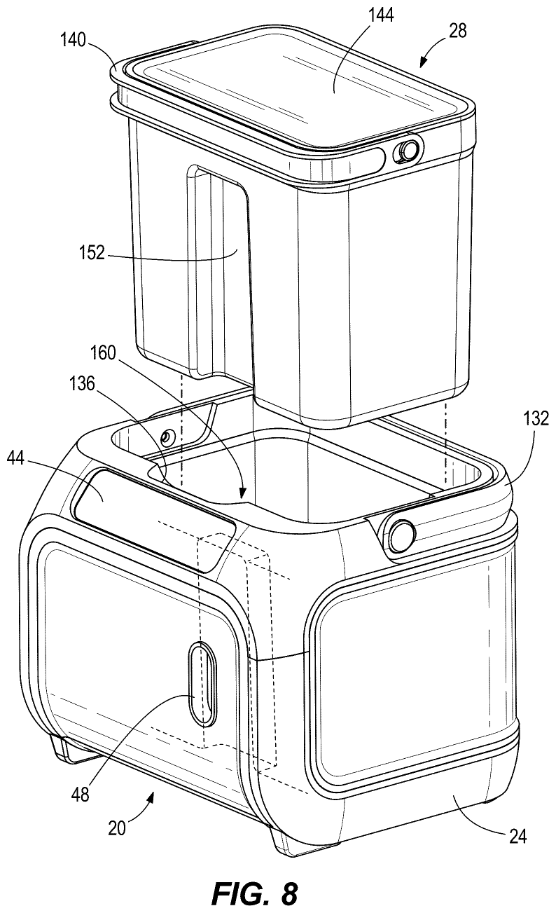

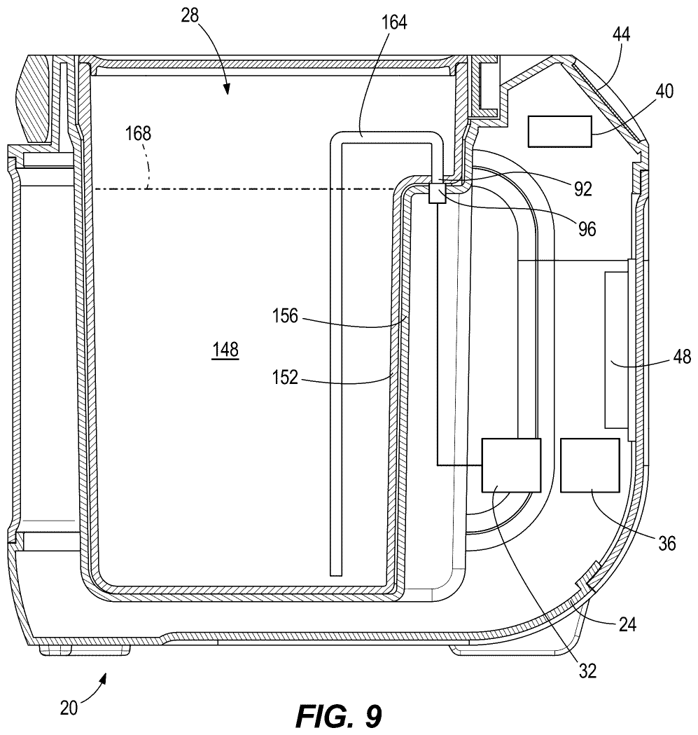

As shown in FIG. 8, the removable fluid reservoir 28 also includes a reservoir lid 144 selectively inhibiting access to a reservoir cavity 148 (see FIG. 9) defined by the walls and floor of the removable fluid reservoir 28. A reservoir recess 152 is formed in the removable fluid reservoir 28 and sized to interact with a corresponding housing projection 156 (see FIG. 9) formed in a housing cavity 160.

As shown in FIG. 9, the reservoir port 92 is formed in a part of the reservoir recess 152 and the housing port 96 is formed in a part of the corresponding housing projection 156. The corresponding shapes of the reservoir recess 152 and the corresponding housing projection 156 cause the removable fluid reservoir 28 to align with the housing cavity 160 so that the reservoir port 92 mates with the corresponding housing projection 156 to seal the internal coolant port. As discussed above, in some implementations, the return line and the output line pass through the internal coolant port. A pickup tube in the form of a reservoir tube 164 connects from the reservoir port 92 to a point below a water line 168 of the reservoir cavity 148. In some implementations, the reservoir tube 164 extends adjacent a floor of the reservoir cavity 148. In some implementations, the reservoir tube 164 is formed in the removable fluid reservoir 28. In some implementations, the reservoir tube 164 is removable similar to a straw.

The positive displacement fluid pump 32, the fluid compressor 36, and the controller 40 are positioned within the housing 24 and fluidly isolated from the removable fluid reservoir 28. Empty cavities of the housing 24 are filled with insulation. In some implementations, an open cell foam provides insulation to the housing 24. In some implementations closed cell foam or another insulation is utilized.

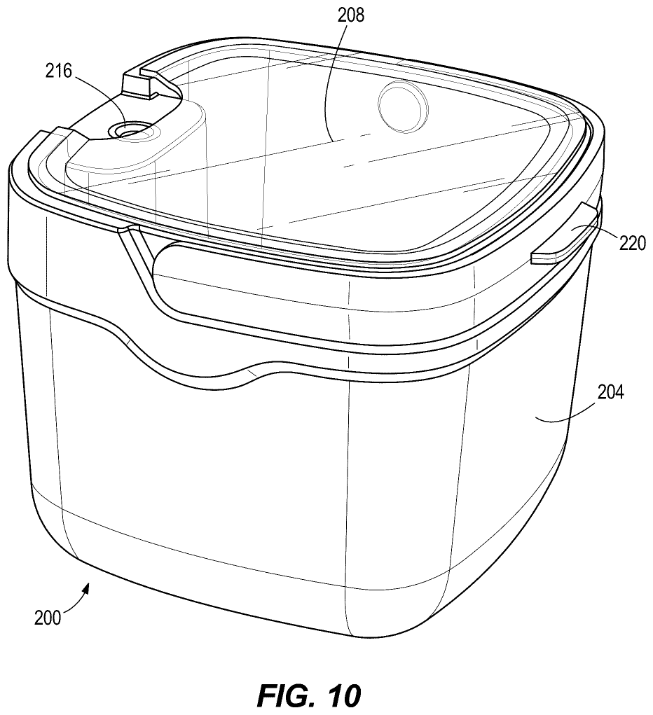

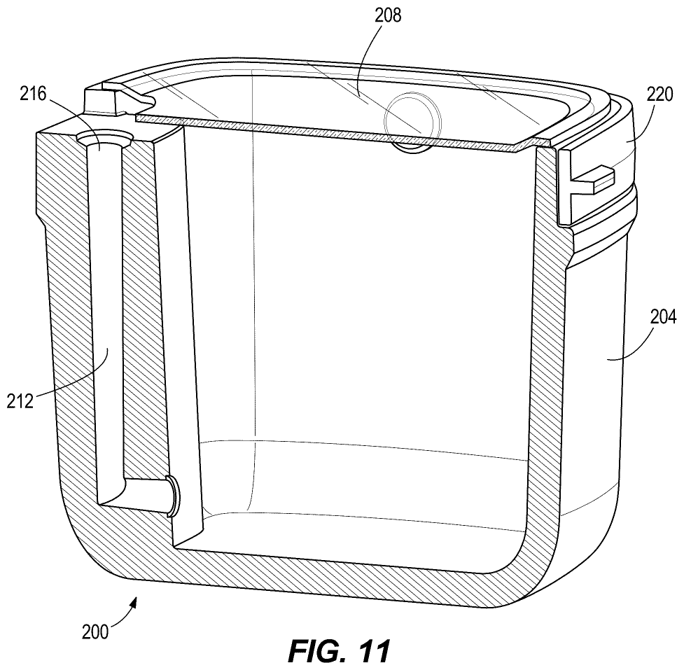

As shown in FIGS. 10 and 11, an insulated fluid reservoir 200 is structured to function with an industrial thermal therapy system including external pumps and compressors. The insulated fluid reservoir 200 is easily fillable with ice and water (or other coolants/fluids) and easily connected to the thermal therapy system. The insulated fluid reservoir 200 includes insulated walls and floor 204, a lid 208, an internal reservoir tube 212 formed in the wall 204, and a reservoir port 216. A reservoir handle 220 is pivotably connected to the walls 204.

For purposes of this description, certain advantages and novel features of the aspects and configurations of this disclosure are described herein. The described methods, systems, and apparatus should not be construed as limiting in any way. Instead, the present disclosure is directed toward all novel and nonobvious features and aspects of the various disclosed aspects, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

Features disclosed in this specification (including any accompanying claims, abstract, and drawings), and/or all of the steps of any method or process so disclosed, may be combined in any combination, except combinations where at least some of such features and/or steps are mutually exclusive. The claimed features extend to any novel one, or any novel combination, of the features disclosed in this specification (including any accompanying claims, abstract, and drawings), or to any novel one, or any novel combination, of the steps of any method or process so disclosed.

As used in the specification and the appended claims, the singular forms “a”, “an”, and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about”, it will be understood that the particular value forms another aspect. It will be further understood that the endpoints of each of the ranges are significant both in relation to the other endpoint, and independently of the other endpoint. The terms “about” and “approximately” are defined as being “close to” as understood by one of ordinary skill in the art.

The terms “coupled”, “connected”, and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic. For example, circuit A communicably “coupled” to circuit B may signify that the circuit A communicates directly with circuit B (i.e., no intermediary) or communicates indirectly with circuit B (e.g., through one or more intermediaries).

Certain terminology is used in the following description for convenience only and is not limiting. The words “right”, “left”, “lower”, and “upper” designate direction in the drawings to which reference is made. The words “inner” and “outer” refer to directions toward and away from, respectively, the geometric center of the described feature or device. The words “distal” and “proximal” refer to directions taken in context of the item described and, with regard to the instruments herein described, are typically based on the perspective of the practitioner using such instrument, with “proximal” indicating a position closer to the practitioner and “distal” indicating a position further from the practitioner. The terminology includes the above-listed words, derivatives thereof, and words of similar import.

Throughout the description and claims of this specification, the word “comprise” and variations of the word, such as “comprising” and “comprises”, means “including but not limited to”, and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the form disclosed. Many modifications and variations will be apparent to those of ordinary skill in the art without departing from the scope and spirit of the present disclosure.

What is claimed is:

1. A cold compression system comprising:
a housing including a compressor outlet, a coolant outlet, a cavity, and an internal coolant port in fluid communication with the coolant outlet;
a positive displacement fluid pump supported by the housing and arranged in fluid communication between the internal coolant port and the coolant outlet;
one or more processing circuits supported by the housing and including one or more memory devices coupled to one or more processors, the one or more memory devices configured to store instructions thereon that, when executed by the one or more processors, cause the one or more processors to selectively activate the positive displacement fluid pump;
a removable fluid reservoir sized to fit within the cavity, the removable fluid reservoir including:
a reservoir defining a water fill line, and
a reservoir port positioned above the water fill line and structured to selectively fluidly couple with the internal coolant port of the housing; and
a cold compression pad including a compression chamber selectively fluidly coupled to the compressor outlet of the housing, and a heat exchanger selectively fluidly coupled to the coolant outlet of the housing.

2. The cold compression system of claim 1, further comprising a user interface in communication with the one or more processing circuits,
wherein the one or more memory devices are further configured to store instructions thereon that, when executed by the one or more processors, cause the one or more processors to:
receive temperature information from the user interface; and
control operation of the positive displacement fluid pump based on the temperature information.

3. The cold compression system of claim 2, further comprising:
a fluid compressor supported by the housing and arranged in fluid communication with the compressor outlet,
wherein the one or more memory devices are further configured to store instructions thereon that, when executed by the one or more processors, cause the one or more processors to receive pressure information from the user interface, and control operation of the fluid compressor based on the pressure information.

4. The cold compression system of claim 1, wherein the removable fluid reservoir further includes thermal insulation adjacent the reservoir.

5. The cold compression system of claim 1, wherein the housing includes a housing carry handle, wherein the removable fluid reservoir includes a reservoir carry handle, and the housing includes a reservoir handle recess sized to receive the reservoir carry handle.

6. The cold compression system of claim 1, wherein the housing includes a housing carry handle movable between a housing handle stowed position adjacent a first side of the housing and a housing handle deployed position, and
wherein the removable fluid reservoir includes a reservoir carry handle movable between a reservoir handle stowed position adjacent a second side of the housing opposite the first side of the housing and a reservoir handle deployed position.

7. The cold compression system of claim 1, wherein the housing includes a housing projection and the internal coolant port is positioned on the housing projection, and
wherein the removable fluid reservoir includes a reservoir recess sized to receive the housing projection and the reservoir port is positioned on the reservoir recess.

8. The cold compression system of claim 1, wherein the housing includes a housing projection, and
wherein the removable fluid reservoir includes a reservoir recess sized to receive the housing projection and inhibit movement of the removable fluid reservoir within the housing.

9. The cold compression system of claim 1, wherein at least one of the internal coolant port or the reservoir port includes a valve that allows fluid flow when the internal coolant port and the reservoir port are coupled together and inhibit flow when the internal coolant port and the reservoir port are decoupled.

10. The cold compression system of claim 1, wherein the internal coolant port engages the reservoir port vertically along a valve axis.

11. The cold compression system of claim 1, wherein the removable fluid reservoir includes a pickup tube fluidly coupled to the reservoir port and extending below the water fill line.

12. The cold compression system of claim 11, wherein the pickup tube is formed within the reservoir.

13. The cold compression system of claim 1, wherein the removable fluid reservoir includes a lid selectively covering the reservoir.

14. The cold compression system of claim 1, wherein the removable fluid reservoir is removable from the housing with the cold compression pad connected to the housing.

15. A system comprising:
a housing including a compressor outlet, a coolant outlet, a cavity, and an internal coolant port in fluid communication with the coolant outlet;
a fluid compressor supported by the housing and arranged in fluid communication with the compressor outlet;
a positive displacement fluid pump supported by the housing and arranged in fluid communication between the internal coolant port and the coolant outlet;
one or more processing circuits supported by the housing and including one or more memory devices coupled to one or more processors, the one or more memory devices configured to store instructions thereon that, when executed by the one or more processors, cause the one or more processors to selectively activate the fluid compressor or the positive displacement fluid pump; and
a removable fluid reservoir sized to fit within the cavity, the removable fluid reservoir including:
a reservoir defining a water fill line, and
a reservoir port positioned above the water fill line and structured to selectively fluidly couple with the internal coolant port of the housing.

16. The system of claim 15, wherein at least one of the internal coolant port or the reservoir port includes a valve that allows fluid flow when the internal coolant port and the reservoir port are coupled together and inhibit flow when the internal coolant port and the reservoir port are decoupled.

17. The system of claim 15, wherein the removable fluid reservoir includes a pickup tube fluidly coupled to the reservoir port and extending below the water fill line.

18. The system of claim 15, wherein the housing includes a housing projection and the internal coolant port is positioned on the housing projection, and
wherein the removable fluid reservoir includes a reservoir recess sized to receive the housing projection and the reservoir port is positioned on the reservoir recess.

19. A bucket configured for use with a cold compression device, the bucket comprising:
at least one side wall comprising an insulation material;
a reservoir defined by the at least one side wall the reservoir further defining a water fill line; and
a reservoir port positioned above the water fill line and structured to selectively fluidly couple with an internal coolant port of a housing of the cold compression device.

20. The bucket of claim 19, wherein the bucket further comprises a pickup tube fluidly coupled to the reservoir port and extending below the water fill line.

21. The bucket of claim 20, wherein the pickup tube is formed within the at least one side wall.

22. The bucket of claim 19, wherein the bucket includes a reservoir carry handle movable between a reservoir handle stowed position adjacent a first side of the housing opposite a second side of the housing and a reservoir handle deployed position.
